(19) United States
(12) Patent Application Publication
(10) Pub. No.: US 2008/0206354 A1
(43) Pub. Date: Aug. 28, 2008

(54) Composition for Improving Hair Growth Comprising an Non-Polar Solvent-Soluble Extract of Cantharis as an Active Ingredient

(75) Inventors: Sang-Cheol Chi, Gyounggi-do (KR); Sung-Yong Kim, Seoul (KR)

Correspondence Address: ROTHWELL, FIGG, ERNST & MANBECK, P.C., 1425 K STREET, N.W., SUITE 800, WASHINGTON, DC, 20005, US

(73) Assignee: Kwang Dong Pharm Co., Ltd., Seoul (KR)

(21) Appl. No.: 11/814,437

(22) PCT Filed: Jan. 18, 2006
(86) PCT No.: PCT/KR06/00195

§ 371 Date: Jul. 20, 2007

(30) Foreign Application Priority Data
Jan. 20, 2005 (KR) 10-2005-0005234

(51) Int. Cl. A61K 35/64; A61Q 7/00

(52) U.S. Cl. 424/541

(57) ABSTRACT

Disclosed herein is a hair growth-stimulating composition containing a non-polar solvent-soluble extract fraction of Cantharis as an active ingredient. The composition shows an excellent hair growth activity with minimal skin irritation compared to conventional ones. It can be used for hair growth in various fields, including medical and non-medical supplies and cosmetics.

### TECHNICAL FIELD

The present invention relates to a hair growth-stimulating composition containing a non-polar solvent extract fraction of Cantharis as an active ingredient.

### BACKGROUND ART

Causes of alopecia, that is, hair loss, are known to include genetic factors, hyper-secretion of the male hormone testosterone, hypersecretion of sebum, a decrease in blood flow in hair follicles, stress, diet, air pollution, hair conditions, etc. Representative of the hair loss preventing agents developed thus far is finasteride (U.S. Pat. No. 5,547,957). It is a specific inhibitor of 2, 5 alpha-reductase, which is the enzyme that metabolizes the androgenic testosterone into dihydrotestosterone (DHT), a major cause of androgenetic alopecia. The compound has been commercialized with the brand name of “PROPECIA” by Merck Co., U.S.A. and has been approved by US FDA. However, this tablet must be taken continually in order to maintain the effect of hair-loss treatment. Further, pregnant women should not handle PROPECIA because of the possibility of absorption. Also, in clinical studies for PROPECIA, men have been reported to experience certain sexual side effects, such as less desire for sex, erectile dysfunction or a decrease in the amount of semen when they took it for a long period of time.

Minoxidil is a direct vasodilator introduced originally for the treatment of hypertension. After it was found that minoxidil has a side effect of hair growth, its topical solution has been commercialized as a hair loss treatment (Korean Pat. No. 10-2003-0020446, U.S. Pat. Nos. 4,596,812; 4,139,619). The effectiveness in growing new hair of minoxidil is known to be attributable to a complicated pharmaceutical mechanism. Minoxidil is the only topical medication approved by the US FDA to stimulate the growth of hair, but has side effects such as skin irritation, itching, blushing, aggravation of hair loss, etc. Likewise, this medication must be continually used in order to maintain its hair growth effect.

Cantharis is a brilliant green soft-bodied blister beetle ,a beetle of the family Meloidae, which includes Epicauta gorhami Marseue, Lytta vesicaforia Latreille, Mylabris cichorii Linne, and Mylabris phalerata Pallas, etc. The term cantharis is also applied to the toxic powdered beetle preparation used as a diuretic, counterirritant and aphrodisiac, containing cantharidin with a minor content of lipids and aromatics. Cantharis is usually applied only topically for limited use, since it induces very high toxicity after oral administration. It is applied to a skin spot of blood stasis or extravasated blood (Herbology, pp 646-647, published in 1991 by Young Lim Publishing Company, Korea).

Korean Pat. Laid-Open Publication No. 2003-0070780 discloses an herbal composition containing several herbal medicines including Cantharis and an anti-inflammatory plaster based thereon. Korean Pat. Laid-Open Publication No. 1995-0007864 discloses a foaming solution prepared by pulverizing Cantharis, in combination with three medicinal herbs, extracting them with alcohol, and filtering the alcohol solution, and a remedy method in which the foaming solution is applied to acupuncture points to form blisters, followed by removing toxic exudates from the blisters.

A hair tonic made from 14 herbal components was introduced in Japanese Pat. Laid-Open Publication No. 2561116. Cantharis is included in this hair tonic. However, it can induce side effect such as skin irritation and even formation of scabs, because Cantharis also has severe toxicity when applied onto skin.

Leading to the invention, intensive and thorough research on hair growth, conducted by the present inventor, resulted in the finding that extract fraction of Cantharis in hexane and non-polar organic solvents has excellent stimulating activity for hair growth, but, low irritation on skin, after screening the pharmacological activity and skin tovicity with extract fractions of Cantharis in various solvents.

### DISCLOSURE OF INVENTION

### Technical Problem

Therefore, it is an object of the the present invention to provide a composition, based on a non-polar solvent-soluble extract fraction of Cantharis, that has an excellent stimulating activity of hair growth and low side effects on the skin.

### Technical Solution

In accordance with an aspect of the present invention, a composition for stimulating hair growth, containing a non-polar solvent-soluble extract fraction of Cantharis as an effective ingredient.

Cantharis useful in the present invention is selected from among Epicauta gorhami Marseue, Lytta vesicaforia Latreille, Mylabris cichorii Linne, Mylabris phalerata Pallas, etc. and combinations thereof.

The non-polar solvent useful for the extraction of Cantharis is selected from among hexane, acetone, chloroform, dichloromethane and ethylacetate, with preference for hexane.

Preferably, the extract of cantharis with the non-polar solvent is contained in an amount from 0.001 to 10 parts by weight based on 100 parts by weight of the composition.

Before extraction, Cantharis is dried using conventional method. Dried Cantharis or its powder is extracted in an organic solvent according to a typical method.

The non-polar solvent-soluble extract fraction of Cantharis contained in the composition of the present invention may be prepared by (1) finely slicing or milling dry Cantharis, (2) extracting it, once or several times, with about 2 to 30 parts by weight of water, a lower alcohol having one to four carbon atoms, or a combination thereof, using cold precipitation, ultrasonication, or reflux cooling, followed by filtering, vacuum concentrating or drying the extract to produce a crude extract and (3)adding a suspension of the crude extract in water to about 1 to 20 volumes of a non-polar solvent, fractioning it once to five times, and concentrating the fractions under vacuum pressure. Optionally, typical fractioning processes may be additionally conducted.

The non-polar solvent-soluble extract fraction of Cantharis prepared in this process was found to have an excellent stimulating activity of hair growth with minimal skin irritation when its effect on hair growth and skin were evaluauted.

Particularly, the hexane-soluble extract fraction of Cantharis shows the most excellent hair growth stimulating effect. Extract fractions with hexane-like organic solvents such as acetone, chloroform, dichloromethane and ethyl acetate also highly stimulate hair growth and induce less skin irritation.

As components of the composition for stimulating hair growth in accordance with the present invention can be prepared using the non-polar solvent-soluble extract fraction of Cantharis and other ingredients. Those are hair growth stimulator or ingredients that can improve the hair growth when applied on scalp topically or any other effective ingredients which have different functions with the above ingredients.

The composition for stimulating hair growth in accordance with the present invention may be formulated for use as medical or non-medical supplies or cosmetics.

Preferably, the composition for stimulating hair growth in accordance with the present invention is formulated into topical dosage forms including medical and non-medical supplies such as liquids, lotions, creams, ointments, gels, aerosols, etc., and cosmetics such as shampoos, shampoo and rinses, hair tonics, hair creams, hair oils, sprays, etc.

For the preparation of the composition for stimulating hair growth in accordance with the present invention, the effective components may be formulated with conventional additives used in medical or non-medical supplies or cosmetics for external use. In detail, solvents such as water, organic solvents, mixed solvents, various solutions, solubilizers, surfactants, penetration enhancers, moisturizing agents, thickeners, pH-adjusting agent, preservatives, stabilizers, buffers, colorants, perfumers, etc., may be used.

Preferably, the hair growth stimulating composition in accordance with the present invention is spread or sprayed directly on the scalp.

The dose of the composition of the present invention can be varied depending on individual conditions such as age, hair loss stage, etc., and formulas. Ususlly, a suitable amount of the composition is applied to the scalp once or several times a day for one week to ones of month.

### BEST MODE FOR CARRYING OUT THE INVENTION

A better understanding of the present invention may be given with the following examples which are set forth to illustrate, but are not to be construed to limit the present invention.

### EXAMPLE

### Composition for Stimulating Hair Growth

1) Preparation of hexane-soluble extract fraction of Cantharis

A solution obtained by the cold-precipitation of 300 g of Cantharis in 2 liters of methanol for 48 hours was filtered and concentrated at reduced pressure. This procedure was repeated three times. About 30g of crude extract of Cantharis was obtained. A suspension of the crude Cantharis extract in 300 [symbol] of purified water was fractioned three times with 300 [symbol] of hexane, and the hexane-soluble fractions were concentrated at a reduced pressure. About 13 g of oily hexane-soluble extract fraction of Cantharis was obtained.

2) Preparation of hair growth stimulating composition for external use

The hexane-soluble extract fraction of Cantharis prepared in Example 1) was dissolved in a mixture of 40% ethanol and Lauroglycol FCC (Gateffosse, France) (9:1) to give a 3% composition for external use. Its pharmacological activity and skin irritation were evaluated as follows. Purified water was used as a control. Minoxyl solution(Hyundai Pharm. Ind. Co., Korea) containing 5% minoxidil was used as a reference 1 and a 1% solution of the crude Cantharis extract of Example 1) in purified water as a reference 2.

2-1) Hair growth

The hair growth activity of the composition prepared in Example 2 was determined using C57BL/6 mice.

After 4-week-old mice were purchased and adapted to a germ-free animal lab for 2 weeks, the hair on the backs of mice were removed, and the mice were stabilized for one day. 8 mice were used in each test group.

The composition according to the present invention, the control, and the references were applied to the hair-removed back of the mice at a dose of 200 [symbol] once a day from the second day after the hair removal until the time of hair nascence as the hair growth cycle of the control group proceeded from catagen to anagen.

The hair-removed area and hair-growth area on the back of mice were photographed a digital camera. The areas in the pictures were digitalized using an image analysis program (Image-pro Express 4.5, Mediacybernetics Co., U.S.A.) Hair growth ratio was calculated as the percentage of hair-grown area to the hair-grown area to the hair-removed area. The results are presented in Table 1, below.

2-2) Skin irritation

The skin irritation by the composition prepared in Example 2 was determined using C57BL/6 mice.

The samples were applied using the same method in Example 2-1). On the 4th day after the application, the degree of skin irritation was evaluated as follows: 0; no irritation, 1; slight erythema, 2; erythema, 3; slight scab, and 4; scab. The results are presented in Table 1, below.

As is apparent from the data of Table 1, the hair growth activity of composition of the present invention was excellent. Its hair growth ration was higher than two times compared to that of minoxidil of the reference 1. However, it did not show any significant difference in skin irritation compared to the control group.

The crude extract of Cantharis of the reference 2 also resulted in excellent hair growth ration compared to minoxidil. But, it induced severe skin irritation, such as scab formation, which makes it unsuitable for external use.

TABLE 1

| | Group | | | |
|---|---|---|---|---|
| Evaluation | Control | Reference1 | Reference2 | Example |
| Hair Growth | 0.08 ± 0.05* | 0.30 ± 0.20 | 0.82 ± 0.34* | 0.68 ± 0.33* |
| Ratio | | | | |
| Skin | 0 | 0.25 ± 0.46 | 3.75 ± 0.46^{‡} | 0.36 ± 0.52 |
| Irritation | | | | |

*Mean ± SD (n = 8)
*Significantly different from reference 1 (P = 0.05)
^{‡}Significantly different from control (P = 0.05)

### MODE FOR THE INVENTION

### Preparation Example 1

### Preparation of Topical Solution for Stimulating Hair Growth 2

A 2% solution for external use was prepared by dissolving a hexane-soluble extract fraction of Cantharis prepared in Example 1 in 40% ethanol.

### Preparation Example 2

### Preparation of Topical Solution for Stimulating Hair Growth 3

A 2% solution for external use was prepared by dissolving a hexane-soluble extract fraction of Cantharis prepared in Example 1) in 10% Transcutol (Gateffosse, France).

### Preparation Example 3

### Preparation of Hair Tonic for Stimulating Hair Growth 1

Hexane-soluble extract
fraction of Cantharis 1 g
Ethanol 40 [symbol]
Camphor 1 g
Vitamin B6 0.2 g
Glycyrrhizic acid 0.1 g
Tocopherol acetate 0.1 g
Perfume 0.2 [symbol]
Purified water q.s. to 100 [symbol]
All ingredients were uniformly mixed and dissolved at room temperature.

### Preparation Example 4

### Preparation of Hair Tonic for Stimulating Hair Growth 2

Hexane-soluble extract
fraction of Cantharis 1 g
Polyethylene glycol 4000 50 [symbol]
L-menthol 1 g
Glycerin 2 g
Perfume 0.2 [symbol]
Polyoxyethylene hydrogenated castor oil 0.5 [symbol]
Purified water q.s. to 100 [symbol]
All ingredients were uniformly mixed and dissolved at room temperature.

### INDUSTRIAL APPLICABILITY

As described hereinbefore, the composition for stimulating hair growth in accordance with the present invention can show an excellent hair growth activity with minimum skin irritation.

Therefore, the composition of the present invention can find various applications for hair growth both in the medical, non-medical and cosmetic fields.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A composition for stimulating hair growth, containing an extract fraction of Cantharis extracted with a non-polar solvent as an active ingredient.

2. The composition as defined in claim 1, wherein the non-polar solvent is selected from the group consisting of hexane, acetone, chloroform, dichloromethane, ethyl acetate and combinations thereof.

3. The composition as defined in claim 1, wherein the extract fraction of Cantharis extracted with the non-polar solvent is contained in an amount from 0.001 to 10 parts by weight based on 100 parts by weight of the composition.

4. The composition as defined in claim 1, wherein the composition is for external use.

5. The composition as defined in claim 4, wherein the composition is formulated for medical or non-medical supplies, or cosmetics.

6. The composition as defined in claim 2, wherein the composition is for external use.

7. composition as defined in claim 3, wherein the composition is for external use.

8. The composition as defined in claim 6, wherein the composition is formulated for medical or non-medical supplies, or cosmetics.

9. The composition as defined in claim 7, wherein the composition is formulated for medical or non-medical supplies, or cosmetics.
